(19) United States
(12) Patent Application Publication
(10) Pub. No.: US 2004/0224914 A1
(43) Pub. Date: Nov. 11, 2004

(54) Method for treatment of angiogenic disorders

(75) Inventors: John K. Jackson, Vancouver (CA); Helen Burt, Vancouver (CA); Christopher Springate, Vancouver (CA); Martin Gleave, Vancouver (CA)

Correspondence Address: OPPEDAHL AND LARSON LLP, P O BOX 5068, DILLON, CO, 80435-5068, US

(73) Assignee: The University of British Columbia, Vancouver (CA)

(21) Appl. No.: 10/828,395

(22) Filed: Apr. 19, 2004

Related U.S. Application Data
(60) Provisional application No. 60/464,160, filed on Apr. 18, 2003.

(51) Int. Cl. A61K 48/00

(52) U.S. Cl. 514/44

(57) ABSTRACT

A therapeutic method for treatment of non-cancerous angiogenesis-related diseases involves administering a therapeutically effective amount of a composition effective to reduce the effective amount of clusterin in the individual. Preferred therapeutic compositions contain antisense oligonucleotides which reduce the effective amount of clusterin.

This application claims the benefit and priority of U.S. Provisional Application No. 60/464,160, filed Apr. 18, 2003, which is incorporated herein by reference.

### BACKGROUND OF THE INVENTION

This application relates to a method for treatment of angiogenic disorders, and in particular non-cancerous angiogenic disorders.

Table 4 provides a non-limiting list of non-cancerous angiogenesis-related diseases and their characteristics. While these diseases have disparate, and in some cases poorly understood causes, they share the common feature of the inappropriate growth of blood vessels, and in many cases, this inappropriate angiogenesis is associated with the significant deleterious symptoms of the disease.

Thus, a therapeutic and therapeutic methodology which reduced or eliminated angiogenesis in individuals suffering from non-cancerous angiogenesis-related diseases would be desirable. It is an object of the present invention to provide such a therapeutic and methodology.

The present invention is based on the surprising finding that reduction in levels of clusterin leads to a reduction in angiogenesis. The glycoprotein clusterin was originally purified from ram rete testes fluid and sertoli cells and was reported to have cell aggregation properties (clustering) at these sites (Blaschuk, Burdzy et al. 1983; Griswold, Roberts et al. 1986). The protein was later found to be associated with Apolipoprotein A1 in plasma and was independently termed apolipoprotein J. Other names for the protein include sulphated glycoprotein-2 (SGP-2), complement cytolysis inhibitor (CCI) and testosterone repressed prostate messenger-2 (TRPM-2). The wide range of names reflects the diversity of tissue distribution and proposed functions for the protein. In fact, the protein has been shown to be present in most human tissues including prostate, testis, epidermis, kidney, uterus, liver spleen and brain and only absent in T lymphocytes (Grima, Zwain et al. 1990). Accordingly, clusterin has been proposed to be involved in many normal physiological functions in the body including lipid transportation (Burkey, Stuart et al. 1992), membrane turnover (Leger, Montpetit et al. 1987), the inhibition of complement induced cytolysis and sperm maturation (Sylvester, Morales et al. 1989). However, the specific mechanism(s) by which this protein functions in normal physiology remains to be elucidated.

An increased expression of clusterin has also been associated with many disease states, including cancer, athereosclerosis, myocardial infarction, kidney disease, and many neurological disorders (Sensibar, Sutkowski et al. 1995). However, it is not known whether the increased expression of clusterin in such diseased tissues is part of the pathophysiology of the disease or merely a reaction to the disease process. Certainly there is a clear relationship between apoptotic cell death and clusterin expression whereby increased amounts of clusterin or clusterin expression (mRNA) are associated with a prosurvival signal in the relevant cells. Originally, it was reported that the increased expression of clustern was associated with cell survival within tissues regressing as a consequence of apoptosis. However, the primary role of clusterin in apoptotic control has been more recently described in many cells. For example, in prostate cancer cells, increased clusterin expression was shown to confer resistance to apoptotic cell death induced by either tumor necrosis factor (TNF-a) or hormone ablation (Sensibar, Sutkowski et al. 1995). In epidermal cancer cells, an increase in clusterin gene expression was shown to confer resistance to apoptotic cell death caused by heat shock and oxidative stress. Similarly, clusterin has been reported to protect granulosa cells from apoptotic cell death during follicular atresia.

The role of clusterin in the circulatory system has come under close scrutiny due to the presence of the protein in vascular endothelial cells, smooth muscle cells in arteries and atrial myocytes in the heart. It has been noted that the expression of clusterin is elevated in tissues undergoing remodeling following injury, such as myocardiocytes close to lesions in the heart. Although the exact role of clusterin in tissue repair is unknown, the protein may induce or promote phenotypic changes rather than general cell proliferation in cells involved in tissue remodeling.

In other cardiovascular diseases, increased clusterin expression in human vascular endothelial cells (HUVEC) is thought to confer resistance to the complement-induced activation of these cells which may be a proinflammatory signal in the pathogenesis of atherosclerosis. Also, the progression of premature vascular and thrombotic disease (atherothrombytic disease) disease is characterized by hyperhomocysteinemia. It is thought that one of the effects of elevated homocysteine levels may be to decrease the levels of the protective protein clusterin in vascular endothelial cells. In arterial graft failure due to anastomotic intimal hyperplasia, vascular endothelial cells are active participants because they migrate over the graft and the injured areas and secrete growth factors for vascular smooth muscle cells, thus contributing to the proinflammatory response at these disease sites. Clusterin expression was shown to be elevated at these disease sites and although clusterin was shown to inhibit the migration and adhesion of endothelial cells, it did not enhance or inhibit cell proliferation.

### SUMMARY OF THE INVENTION

The present invention provides a therapeutic in the form of a composition effective to reduce the effective amount of clusterin in an individual, and to a therapeutic method comprising the steps of administering to an individual suffering from the non-cancerous angiogenesis-related disease a therapeutically effective amount of a composition effective to reduce the effective amount of clusterin in the individual. Preferred therapeutic compositions comprise antisense oligonucleotides which reduce the effective amount of clusterin.

### BRIEF DESCRIPTION OF THE DRAWINGS

FIG. 1 shows cell viability of HUVECS following exposure to antisense in the presence and absence of paclitaxel.

FIG. 2 shows cell viability of HUVECS following exposure to antisense in the presence and absence of camptothecin.

FIG. 3 shows cell viability of HUVECS following exposure to antisense in the presence and absence of doxorubicin.

### DETAILED DESCRIPTION OF THE INVENTION

As used in the specification and claims of this application, the term “clusterin” refers to the glycoprotein originally derived from rat testes, and to homologous proteins derived from other mammalian species, including humans, whether denominated as clusterin or an alternative name. The sequences of numerous clusterin species are known. For example, the sequence of human clusterin is reported by Wong et al., Eur. J. Biochem. 221 (3), 917-925 (1994), and in NCBI sequence accession number NM_{—}001831, and is set forth as Seq. ID. No. 1 with the coding sequence spanning bases 48 to 1397.

As used in the specification and claims of this invention, an oligonucleotide consisting essentially of a specified sequence as reflected by a Seq. ID No. is an oligonucleotide with exactly the same sequence as that listed, or which differs from the exact sequence, for example as a result of the addition or substitution of one or two bases, but retains the ability to act as an antisense or RNAi agent to reduce the effective amount of clusterin. In the case of RNAi agents, the sequences given represents the sense si RNA strand, without the 3′-dTdT sequence, and the term consisting essentially of encompasses sequences including this deoxynucleotide tail.

The present invention provides a therapeutic composition, and methods for using such a composition for prevention of angiogenesis associated with non-cancerous angiogenesis-associated diseases. As used in this application, the term “non-cancerous angiogenesis-associated diseases” refers to non-cancerous diseases or conditions wherein inappropriate angiogenesis is observed as a symptom of the disease. Specific, non-limiting examples of such diseases are those set forth in Table 1.

The therapeutic methods of the invention achieve a reduction in the effective amount of clusterin present in the individual being treated. As used in this application, the “effective amount of clusterin” is the amount of clusterin which is present in a form which is functional to enhance or promote angiogenesis. The effective amount of clusterin may be reduced by decreasing the expression rate of clusterin, increasing the rate of clusterin degradation, or by modifying clusterin (for example by binding with an antibody) such that it is rendered inactive.

Reduction in the effective amount of clusterin may be accomplished by the administration of antisense oligodeoxynucleotides (ODNs), particularly antisense ODNs which are complementary to a region of the clusterin mRNA spanning either the translation initiation site or the termination site. The ODNs employed may be modified to increase the stability of the ODN in vivo. For example, the ODNs may be employed as phosphorothioate derivatives (replacement of a non-bridging phosphoryl oxygen atoms with a sulfur atom) which have increased resistance to nuclease digestion. MOE (2′-O-(2-methoxyethyl) modification (ISIS backbone) is also effective. Construction of such modified ODN is described in detail in U.S. patent application Ser. No. 10/080,794, published as US-0030166591, which is incorporated herein by reference. Specific antisense species which may be used in the method of the invention include, without limitation, those sequences listed in Seq. ID Nos. 2-15. Other antisense species which target expression of clusterin are described in U.S. Pat. No. 6,383,808, which is incorporated herein by reference.

Administration of antisense ODNs can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978 which are incorporated herein by reference. In general, the antisense is administered by intravenous, intraperitoneal, subcutaneous or oral routes, or direct local tumor injection.

Reduction of clusterin may also be accomplished using an RNAi approach. RNA interference or “RNAi” is a term initially coined by Fire and co-workers to describe the observation that double-stranded RNA (dsRNA) can block gene expression when it is introduced into worms (Fire et al. (1998) Nature 391, 806-811, incorporated herein by reference). dsRNA directs gene-specific, post-transcriptional silencing in many organisms, including vertebrates, and has provided a new tool for studying gene function. RNAi involves mRNA degradation, but many of the biochemical mechanisms underlying this interference are unknown. The use of RNAi has been further described in Carthew et al. (2001) Current Opinions in Cell Biology 13, 244-248, and Elbashir et al. (2001) Nature 411, 494-498, both of which are incorporated herein by reference. Clusterin expression can be reduced by the introduction of RNA molecules of about 21 to about 23 nucleotides that direct cleavage of clusterin-specific mRNA to which their sequence corresponds. It is not necessary that there be perfect correspondence of the sequences, but the correspondence must be sufficient to enable the RNA to direct RNAi cleavage of the target mRNA. Specific useful RNA sequence for this purposes are set forth in Seq. ID Nos. 16-23 and sequences complementary thereto.

The RNA molecules of the invention are used in therapy to treat patients, including human patients, that have non-cancerous angiogenisis-related diseases. siRNA molecules of the invention are administered to patients by one or more daily injections (intravenous, subcutaneous or intrathecal) or by continuous intravenous or intrathecal administration for one or more treatment cycles to reach plasma and tissue concentrations suitable for the regulation of the targeted mRNA and protein. The RNAi agent may be introduced as discrete siRNA molecules, or as part of an siRNA expression plasmid that results in the production of the RNAi agent in situ. In the latter case, sequences that contain the stated sequences and a complementary sequence separated by a loop region (for example of 9 bases) such that hairpin structures are formed and subsequently cleaved to form the RNAi agent may be employed.

In accordance with the invention, a therapeutic agent that reduces the effective amount of clusterin is administered to a subject, preferably a human subject, in need of treatment for a non-cancerous angiogenic disorder. The therapeutic agent is administered in an amount effective to result in a reduction of angiogenesis. It will be appreciated by persons skilled in the art that this amount will vary with the specific therapeutic agent, the route of administration and the type of carrier employed, if any. However, the determination of appropriate amounts is a matter of routine experimentation, and is generally defined by an upper limit determined based on toxicity, or a balancing of toxicity and efficacy.

Antisense oligonucleotides may be administered by normal means known to those skilled in the art such as by injection into the blood stream as a solution in an isotonic injection media. The injection regime may be by daily injection of a sufficient dose of the agent to maintain a therapeutic concentration of the oligonucleotide necessary for inhibition of the disease. Other parenteral routes include for example, intramuscular, intraperitoneal and subcutaneous. However, these agents may also be given orally using modern methods, known to those skilled in the art, to protect the oligonuclotides from degradation and enhance the passage of the oligonucleotides from the intestine to the blood stream.

These agents may also be delivered by other means more conducive to effective treatment of the disease. For example it might be better to inject a solution of the antisense oligonucleotide directly into a disease site for example by intraarticular injection of a solution of the oligonucleotide. It is well known that larger (molecular weight) molecules such as proteins and oligonucleotides are cleared rather slowly from the synovial joint so that an extended residence time in the joint may allow greater penetration of the oligonucleotides into the target diseased cells. Also a much higher local concentration of the oligonucleotide may be achieved at the target site as compared to systemic routes of administration allowing for more effective treatment of the disease. Such localized injection methods might be suitable for many other angiogenic diseases such as injection and around keloids, directly into the eye, around vascular implants, around surgical trauma sites or into sites of psoriasis inflammation.

Controlled release drug delivery systems are particularly applicable to the effective treatment of various of the angiogenic related diseases and the following examples illustrate the applicability of these systems. For angiogenic diseases of the eye the oligonucleotides may be administered directly onto the eye suspended in a biocompatible polymeric matrix such as a gel that released the agent in a controlled manner. The oligonucleotides might be encapsulated in microspheres made from, for example, poly lactic co glycolic acid and injected into target sites so that the oligonucleotides released from the microspheres by diffusion or as the biodegradable matrix broke down. Such microspheres might also be injected directly into the arthritic joint to allow for entrapment of the oligonucleotides in the joint where they might release over a period of hours to days to months depending on the therapeutic need. Viscous gels such as those made from hyaluronic acid might be utilized for this purpose since this agent is mucoadhesive and may allow the oligonucleotide to be localized on the appropriate tissues, such as, for example around the site of placement of a vascular graft or around a surgical trauma site (to prevent surgical adhesions). The positively charged biocompatible and biodegradable polysaccharide chitosan has been shown to be useful in binding and delivering oligonucleotides in vivo and this agent might be included in injectable formulations to allow for the controlled release at the site of the disease.

The therapeutic agent that reduces the effective amount of clusterin may be administered individually, or in combination with other compositions that inhibit angiogenesis (capillary growth), in either order or concurrently. Such compositions include, without limitation, antiproliferative drugs such as taxanes (e.g. paclitaxel), camptothecin and anti-angiogenic derivatives thereof, and doxorubicin which inhibit Huvecs in the low nanomolar range by the induction of apoptosis. As reflected in the results below, inhibition of cell proliferation induced by these antiproliferative drugs was enhanced by administration of antisense to produce downregulation of clusterin.

The invention will now be further described with reference to the following non-limiting example.

### EXAMPLE

HUVECS were grown for 2 days in wells after seeding at 1200 per well. Antisense oligonucleotide of Seq. ID No. 5 (4 μg/ml with LIPOFECTIN™) was added in serum free medium and incubated with the cells for 4 hours. Then 100 μl of serum was added and incubation was continued overnight. The next day, 150 μl of drug solution in serum medium was added. After two days, 20 μl of mts solution was added and left for approximately 3 hours. Cell viability was determined as the difference between absorption at 490 and 595 nm.

Table 1 shows the measured absorbances for a first series of experiments in the which the drug tested was paclitaxel. The first row of results is the absorbance at 490 nm. The second row of results is the absorbance at 595 nm.

TABLE 1

| | | | | | | | | | Drug |
|---|---|---|---|---|---|---|---|---|---|
| | | | | MM | | Drug | Drug | Drug | MM |
| Control | AS 50 nM | AS 100 nM | AS 200 nM | 200 nM | Drug | AS 50 nM | AS 100 nM | AS 200 nM | 200 nM |
| 0.75 | 0.66 | 0.40 | 0.28 | 0.66 | 0.53 | 0.50 | 0.24 | 0.17 | 0.21 |
| 0.05 | 0.04 | 0.09 | 0.07 | 0.10 | 0.12 | 0.09 | 0.04 | 0.05 | 0.01 |

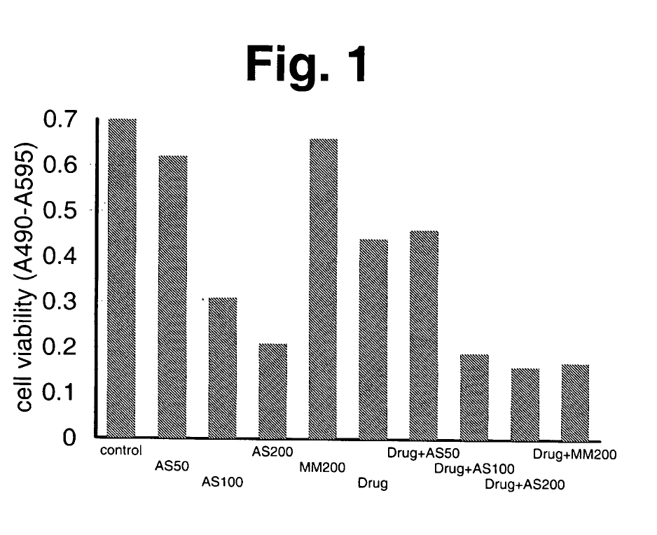

FIG. 1 shows the cell viability for each test sample in this set graphically. The bar in the center represents a mismatch (MM) control used at 200 nM. As shown, a dose dependent response to antisense concentration is observed, and the response is greater in the presence of 100 nM paclitaxel.

Table 2 shows the measured absorbances for a first series of experiments in the which the drug tested was camptothecin. The first row of results is the absorbance at 490 nm. The second row of results is the absorbance at 595 nm.

TABLE 2

| | | | | | | | | | Drug |
|---|---|---|---|---|---|---|---|---|---|
| | | | | MM | | Drug | Drug | Drug | MM |
| Control | AS 50 nM | AS 100 nM | AS 200 nM | 200 nM | Drug | AS 50 nM | AS 100 nM | AS 200 nM | 200 nM |
| 0.67 | 0.66 | 0.39 | 0.21 | 0.58 | 0.55 | 0.53 | 0.31 | 0.15 | 0.33 |
| 0.10 | 0.10 | 0.06 | 0.04 | 0.13 | 0.07 | 0.07 | 0.08 | 0.05 | 0.04 |

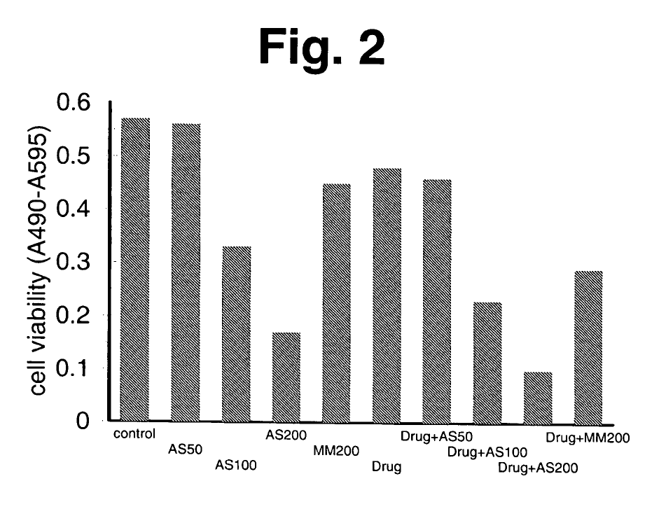

FIG. 2 shows the cell viability for each test sample in this set graphically. The bar in the center represents a mismatch (MM) control used at 200 nM. As shown, a dose dependent response to antisense concentration is observed, and the response is greater in the presence of 100 nM camptothecin.

Table 3 shows the measured absorbances for a first series of experiments in the which the drug tested was doxorubicin. The first row of results is the absorbance at 490 nm. The second row of results is the absorbance at 595 nm.

TABLE 3

| | | | | | | | | | Drug |
|---|---|---|---|---|---|---|---|---|---|
| | | | | MM | | Drug | Drug | Drug | MM |
| Control | AS 50 nM | AS 100 nM | AS 200 nM | 200 nM | Drug | AS 50 nM | AS 100 nM | AS 200 nM | 200 nM |
| 0.76 | 0.66 | 0.35 | 0.22 | 0.53 | 0.74 | 0.66 | 0.32 | 0.18 | 0.39 |
| 0.04 | 0.07 | 0.05 | 0.04 | 0.08 | 0.08 | 0.06 | 0.04 | 0.02 | 0.03 |

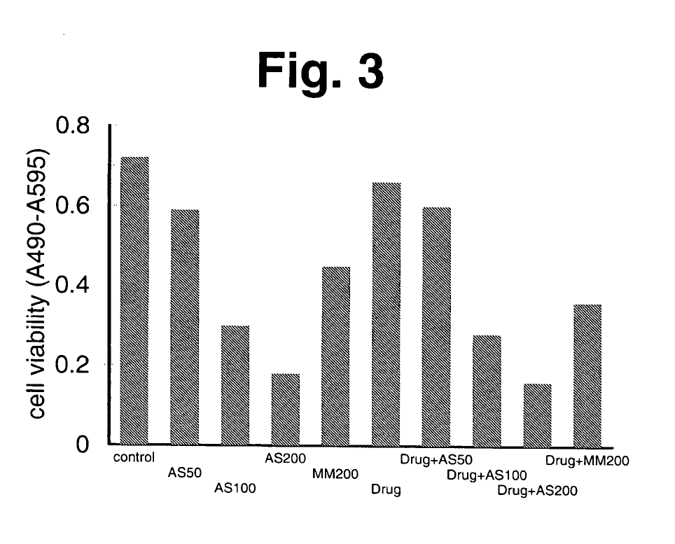

FIG. 3 shows the cell viability for each test sample in this set graphically. The bar in the center represents a mismatch (MM) control used at 200 nM. As shown, a dose dependent response to antisense concentration is observed, and the response is greater in the presence of 200 nM doxorubicin.

TABLE 4

| Angiogenesis Related | |
|---|---|
| Disease | Disease Description |
| Atherosclerotic plaque | A buildup of cholesterol and fatty material within a blood vessel |
| growth and hemorrhage | due to the effects of atherosclerosis. The progressive narrowing and |
| | hardening of the arteries over time. This is known to occur to some |
| | degree with aging, but other risk factors that accelerate this process |
| | have been identified. These factors include: high cholesterol, high |
| | blood pressure, smoking, diabetes and family history for |
| | atherosclerotic disease. |
| Chronic cystitis | Inflammation of the urinary bladder. |
| Crohn's disease | An inflammatory disease of the gastrointestinal tract that seems to |
| | have both genetic and environmental causes, not well understood. |
| | The peak incidence of onset of this disease is between 15 and 25 |
| | years of age. Crohn's also occurs in later years between the ages of |
| | 55 and 60. Common symptoms include recurrent abdominal pains, |
| | fever, nausea, vomiting, weight loss and diarrhoea which is |
| | occasionally bloody. Complications include gastrointestinal |
| | bleeding, fistulas and anal fissures. Treatment includes |
| | anti-inflammatory drugs and corticosteroids. Surgery is successful |
| | in a select few. |
| Diabetic retinopathy | A major cause of blindness in diabetics. Retinal disease results |
| | from adverse effects on the blood vessels which supply the retina. |
| | Swollen retinal vessels which leak fluid into the retina are |
| | commonly seen on physical examination of the eyes. Poorly |
| | controlled insulin dependent diabetes and/or hypertension are the |
| | major risk factors. Symptoms include decreased vision and colour |
| | perception. |
| Dystrophic | This represents a group of rare inherited disorders in which |
| epidermolysis bullosa | blistering of the skin occurs in response to skin trauma. Large |
| | fluid-filled blisters can occur in response to injury, skin rubbing, |
| | chafing or even increases in room temperature. Secondary bacterial |
| | infection of the blisters is common. Complications include |
| | oesophageal stricture, infections, loss of function of hands and feet |
| | and malnutrition. The dermatologist is the expert in the evaluation |
| | and treatment of this disorder. |
| Infantile hemangiomas | Tumor-like clusters of proliferating capillaries. Occur in 1 in 100 |
| | births and 1 in 4 premature births |
| Intraperitoneal bleeding | A condition in which tissue more or less perfectly resembling the |
| in endometriosis | uterine mucous membrane (the endometrium) and containing |
| | typical endometrial granular and stromal elements occurs aberrantly |
| | in various locations in the pelvic cavity. |
| Macular degeneration | Breakdown or damage to a portion of the retina known as the |
| | macula. Symptoms include blurring of vision (in central visual |
| | field), colours appear dim and difficulty reading or performing |
| | work up close. |
| Prostate growth in | A benign enlargement of the prostate gland begins normally after |
| benign prostatic | age 50 years probably secondary to the effects of male hormones. If |
| hypertrophy | significant enlargement occurs, it may pinch off the urethra making |
| | urination difficult or impossible. |
| Psoriasis | A common chronic, squamous dermatosis, marked by |
| | exacerbations and remissions and having a polygenic inheritance |
| | pattern. The most distinctive histological findings in well |
| | developed psoriasis are Munro microabscesses and spongiform |
| | pustules. It is characterised clinically by the presence of rounded, |
| | circumscribed, erythematous, dry scaling patches of various sizes, |
| | covered by greyish white or silvery white, umbilicated and lamellar |
| | scales, which have a predilection for the extensor surfaces, nails, |
| | scalp, genitalia and lumbosacral region. Central clearing and |
| | coalescence of the lesions produce a wide variety of clinical |
| | configurations, including annular or circinate, discoid or nummular, |
| | figurate and gyrate arrangements. |
| Rheumatoid arthritis | Chronic inflammatory disease in which there is destruction of |
| | joints. Considered by some to be an autoimmune disorder in which |
| | immune complexes are formed in joints and excite an inflammatory |
| | response (complex mediated hypersensitivity). Cell-mediated (type |
| | IV) hypersensitivity also occurs and macrophages accumulate. This |
| | in turn leads to the destruction of the synovial lining (see pannus). |
| Verruca vulgaris | A keratotic papilloma of the epidermis which occurs most |
| | frequently in young persons as a result of localised infection by |
| | human papilloma virus, usually types 2 and 4; the lesions are of |
| | variable duration, eventually undergoing spontaneous regression, |
| | and are both exophytic and endophytic, with hyperkeratosis, |
| | parakeratosis, hypergranulosis, koilocytosis, and papillomatosis. |
| Surgical adhesions | Surgical adhesions are regions of tissue adhesion following surgery |
| | whereby the traumatized tissues repair and form connective masses |
| | between tissue surfaces that become vacularized via angiogenesis |
| | and are permanent structures that often require secondary |
| | procedures. |
| Keloids | A keloid is a greatly enlarged scar that projects above the skin |
| | surface. |
| Non cancerous lesions | These are benign masses and growths in the body made from |
| | fibrous, non canceous tissues. For example uterine fibroids are |
| | large cyst-like growths in the uterus that become heavily |
| | vascularized. |

1. A method for treatment of a non-cancerous angiogenesis-related disease, comprising the steps of administering to an individual suffering from the non-cancerous angiogenesis-related disease an amount of a therapeutic composition effective to reduce the effective amount of clusterin in the individual.

2. The method of claim 1, wherein the therapeutic composition comprises an antisense oligonucleotide complementary to the sequence of human clusterin (Seq. ID. No. 1).

3. The method of claim 2, wherein the antisense oligonucleotide is selected from the group consisting of oligonucleotides whose sequence consists essentially of a sequence as set forth in Seq. ID Nos. 2-15.

4. The method of claim 1, wherein the therapeutic composition comprises an RNAi agent.

5. The method of claim 4, wherein the RNAi agent is selected from the group consisting of oligonucleotides whose sequence consists essentially of a sequence as set forth in Seq. ID Nos. 16 to 23 or a sequence complementary thereto.

6. A method for reducing angiogenesis in a non-cancerous angiogenesis-related disease, comprising the steps of treating cells of the cancer with amount of a therapeutic composition effective to reduce the effective amount of clusterin in the cells, and thereby to reduce the occurrence of angiogenesis.

7. The method of claim 6, wherein the therapeutic composition comprises an antisense oligonucleotide complementary to the sequence of human clusterin (Seq. ID. No. 1).

8. The method of claim 7, wherein the antisense oligonucleotide is selected from the group consisting of oligonucleotides whose sequence consists essentially of a sequence as set forth in Seq. ID Nos. 2-15.

9. The method of claim 6, wherein the therapeutic composition comprises an RNAi agent.

10. The method of claim 9, wherein the RNAi agent is selected from the group consisting of oligonucleotides whose sequence consists essentially of a sequence as set forth in Seq. ID Nos. 16 to 23 or a sequence complementary thereto.


SEQUENCE LISTING (23 sequences)

SEQ ID NO: 1 - DNA, 1676 bp, human
gaattccgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccacagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcaccttacgggggcatcttgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgaaaaaaaaaaaaaaaaaaaaaaaaaaggaattc

SEQ ID NO: 2 - DNA, 21 bp, human
gcacagcaggagaatcttcat

SEQ ID NO: 3 - DNA, 21 bp, human
gcacagcagcaggatcttcat

SEQ ID NO: 4 - DNA, 21 bp, human
tggagtctttgcacgcctcgg

SEQ ID NO: 5 - DNA, 21 bp, human
cagcagcagagtcttcatcat

SEQ ID NO: 6 - DNA, 21 bp, human
attgtctgagaccgtctggtc

SEQ ID NO: 7 - DNA, 21 bp, human
ccttcagctttgtctctgatt

SEQ ID NO: 8 - DNA, 21 bp, human
agcagggagtcgatgcggtca

SEQ ID NO: 9 - DNA, 21 bp, human
atcaagctgcggacgatgcgg

SEQ ID NO: 10 - DNA, 21 bp, human
gcaggcagcccgtggagttgt

SEQ ID NO: 11 - DNA, 21 bp, human
ttcagctgctccagcaaggag

SEQ ID NO: 12 - DNA, 21 bp, human
aatttagggttcttcctggag

SEQ ID NO: 13 - DNA, 21 bp, human
gctgggcggagttgggggcct

SEQ ID NO: 14 - DNA, 18 bp, human
tctcccggcttgcgccat

SEQ ID NO: 15 - DNA, 18 bp, human
tctcccggcatggtgcat

SEQ ID NO: 16 - DNA, 19 bp, human
ccagagcucgcccuucuac

SEQ ID NO: 17 - RNA, 19 nt, human
gaugcucaacaccuccucc

SEQ ID NO: 18 - RNA, 19 nt, human
cuaauucaauaaaacuguc

SEQ ID NO: 19 - RNA, 17 nt, artificial
  clusterin targeted siRNA sequence
acaguuuuguugaauua

SEQ ID NO: 20 - RNA, 18 nt, human
augaugaagacuguccgc

SEQ ID NO: 21 - RNA, 20 nt, human
ugaaugaagggacuaaccug

SEQ ID NO: 22 - RNA, 20 nt, human
cagaaauagacaaagugggg

SEQ ID NO: 23 - RNA, 20 nt, human
acagagacuaagggaccaga